(19) United States
(12) Patent Application Publication
(10) Pub. No.: US 2007/0099174 A1
(43) Pub. Date: May 3, 2007

(54) Rapid Testing for Nutrients

(76) Inventors: Bob Han, San Francisco, CA (US); Xiaoliang Han, San Francisco, CA (US)

Correspondence Address: Xiaoliang Han, 1671 11th Ave, San Francisco, CA, 94122, US

(21) Appl. No.: 11/163,926

(22) Filed: Nov. 3, 2005

(51) Int. Cl. C12Q 1/00; C12Q 1/68; G01N 33/92; C12M 1/34

(52) U.S. Cl. 435/4; 435/6; 436/71; 436/90; 435/287.1

(57) ABSTRACT

This invention presents the rapid testing for nutrients in products including, but not limited, medicines, food supplements or additives, cosmetics, drinks, fruits, vegetables, and etc. The rapid testing is performed on strips coated with indicators. Nutrients such as Vitamin C can be detected in very small volume. The detection is semi-quantitative by naked eye or becomes quantitative with help of a hand-held device. The rapid testing in this invention can be very easily performed by individuals at most places, such as home, office, market, school, hospital, laboratories and etc. The rapid testing does not require expensive instruments, chemicals, or professional trainings.

### BACKGROUND OF THE INVENTION

This invention presents the rapid testing for nutrients in products including, but not limited, medicines, food supplements or additives, cosmetics, drinks, fruits, vegetables, and etc. Rapid testing for nutrients is very useful in maintenance and improvement of health of living organisms, including human, on the earth. Adequate nutrition is closely related to the human health in both developed countries and developing countries. Validation of nutrients in terms of amount and composition is a keystone to reach adequate nutrition. There are existing methods to validate nutrients by utilizing expensive instruments and complicated chemicals. These methods can only be operated in laboratories by professionals. Therefore, consumers can only rely on the published testing data or read the labels to learn the nutrition facts. A simple and rapid testing for nutrients will allow consumers to validate the nutrient by themselves and help them to reach adequate nutrition.

The rapid testing in this invention can be very easily performed by individuals at most places, such as home, office, market, school, hospital, laboratories and etc. The rapid testing does not require expensive instruments, chemicals, or professional trainings.

### SUMMARY OF THE INVENTION

This invention presents the rapid testing for nutrients in products including, but not limited, medicines, food supplements or additives, cosmetics, drinks, fruits, vegetables, and etc. The rapid testing is performed on strips coated with indicators. Nutrients such as Vitamin C can be detected in very small volume. The detection is semi-quantitative by naked eye or becomes quantitative with help of a hand-held device. The rapid testing in this invention can be very easily performed by individuals at most places, such as home, office, market, school, hospital, laboratories and etc. The rapid testing does not require expensive instruments, chemicals, or professional trainings.

### BRIEF DESCRIPTION OF DRAWINGS AND FIGURES

FIG. 1. The Rapid Testing for Nutrient in Medicine, Cosmetics, Drinks, Fruits, and Vegetables

2 micro liters of samples from corresponding products was applied on the rapid testing strips. Results were read after 2 min. Amount of vitamin C in medicine, cosmetic product, juice, Lime and tomato can be estimated by naked eye. Orange contains much less amount of vitamin C than lime.

FIG. 2. Testing rang and sensitivity

Rapid testing of vitamin C from a serial diluted vitamin C standard solution. The minimum testing concentration is 0.01%. The minimum detectable amount of vitamin C in 2 micro liters is 0.2 micro grams.

FIG. 3. Rapid Testing for Vitamin C in Different Brand of Products for Vitamin Supplement

2 ul of samples from corresponding products was applied on the rapid testing strips. Results were read after 5 to 10 min. Amount of vitamin C in different brand of nutrient supplements can be estimated by naked eye. Some brand of nutrient supplements may not contain the correct amount of vitamin C as label claimed. Optimization of materials in the rapid testing will achieve better results.

FIG. 4. Supporting Materials for Rapid Testing

Filter paper (Lane A) and poster (Lane B) as supporting material for rapid testing shown different shapes of spots whereas the testing range and sensitivity are similar.

FIG. 5. Concentration of Indicator for Rapid Testing

Different concentrations of indicator were coated on supporting materials of rapid testing. 2× of indicator shown better result than 1× and 4× of indicator coated. Optimization of indicator concentration and processes applied in the rapid testing will achieve better results.

FIG. 6. Volume of Samples for Rapid Testing

Testing samples applied on rapid testing shown a little difference in testing results when different concentration of indicator were coated on testing strips (Panel A, 2×; and Panel B, 4×). Large volume of sample shown stronger signals. Rapid testing strips also can dip into samples to perform the testing if enough samples available (Panel C).

FIG. 7. Specificity of Rapid Testing for Nutrient

Common influencing substances such as acid, base and oxide substances were tested on rapid testing. As shown in Panel A, 100 mM Hydrochloride Acid (HCl) and Citric Acid (CA) shown a very little signal comparing with the vitamin C at the similar concentration in Panel C. 100 mM Sodium Hydroxide (NaOH) and 30% Hydroperoxide (H2O2) did not see any signal on rapid testing strips.

### DETAILED DESCRIPTION OF THE INVENTION

Testing or detection of nutrients in products is mostly conducted in laboratories well equipped with expensive instrument and chemicals. The people operated the testing or detection requires professional training. It is impossible for general population to access these testing and detections. However, it will greatly benefit consumers if they are provided with simple and rapid testing for nutrients, which will guide them to make correct choice in case they do not know the nutrient fact of the products, or the label of products did not match the real content of products.

There are many nutrients in the products consumed by human. There also have different method to detect each of nutrients. There even are many different methods to detect the same nutrient. Usually the samples containing nutrients are not ready to be tested or detected. Therefore, many different procedures and process for sample preparation make the nutrient testing even more complicated.

Nutrients consumed by human include protein, fat, carbohydrates, vitamins, minerals, and etc. Each nutrient is detected by different methods and procedure. The methods to detect these nutrients include chemical reactions, physical procedures, biological process, High Performance Liquid Chromatography (HPLC), Gas Chromatography (GC), Mass Spectrometry (MS), gel electrophoresis. Some of these methods need to be performed in well equipped laboratories, some of the methods demand expensive equipments, and some of the methods require well trained professionals to perform the detection.

The same nutrient can be detected by different methods depended on formulation of nutrient and application of the nutrient. For examples, the method to detection of pure vitamin C is different from the methods to detect formulated vitamin C as medicine. The pure vitamin C can be detected with either by infrared absorption with an expensive instrument or by chemical reaction with alkaline cupric tartrate in laboratories. Quantitation of pure vitamin C involves more complicated chemical reaction, such as mixing vitamin C with sulfuric acid and starch, then titrating with iodine.

There are three formulations of vitamin C as medicine and each formulation are tested with different methods also. Three formulations of vitamin C as medicine are injection solution formulation, oral solution formulation and tablets formulation. To detect vitamin C in injection formulation or oral solution, add trichloroacetic acid and activated charcoal into injection solution, filter through a fluted filter a few times until clear. Add pyrrole in to filtrate and heat at 50° C. Vitamin C is present if solution developed blue color. Vitamin C in injection solution or oral solution can also be detected by Gas Chromatography and it respond to the flame test for sodium. To detect vitamin C in tablets, the tables need to be processed and prepared for the testing. The finely powdered tables are triturated with sufficient diluted alcohol. The solution is filtered and filtrate is used for detection as injection solution.

Quantitation of vitamin C in about medicines is conducted by different methods also. One method involves mixing vitamin C solution with metaphosphoric-acetic acids and titrating with standard dichlorophenol-indophenol solution until a rose-pink color persists for at least 5 seconds. The other method recommended for vitamin injection solution is more complicated and instrument demanding. There are five steps in the method, mobile phase, standard preparation, assay preparation, high performance liquid chromatography, and procedure. The mobile phase consists of dibasic sodium phosphate and monobasic potassium phosphate, adjusting pH to 2.5 with phosphoric acid. Dissolve an accurately weighed quantity of USP vitamin C RS in mobile phase to obtain a Standard Solution with a known concentration of vitamin C about 0.5 mg/ml. For assay preparation, dilute vitamin C containing solution quantitatively with Mobile phase to obtain a Vitamin C Solution having a concentration of vitamin C about 0.5 mg/ml. The high performance liquid chromatography is equipped with a 245-nm detector and a 6 mm×150 mm column that contains packing L39. The flow rate is about 0.6 ml per minute. Chromatograph the Standard Solution first and record the peak responses as directed under procedure. The column efficiency is not less than 3500 theoretical plates, the tailing factor is nor more than 1.6 and the relative standard deviation for replicated Vitamin Solution is not more than 1.5%.

The procedure for the high performance liquid chromatography includes separately injection of equal volume (about 4 ul) of the Standard Solution and Vitamin C Solution in to chromatograph, record the chromatograms, and measure the responses for the major peak. Calculate the quantity, in mg, of vitamin C in each ml of the Vitamin C Solution taken by the formula: CD(rᵤ/rₛ). in which C is the concentration, in mg per ml of vitamin C in Standard Solution, D is the dilution factor, and rᵤ and rₛ are the peak response obtained from the Vitamin C Solution and the Standard Solution respectively.

It is beyond the scope here to list and detail all the different methods to test or detect all different nutrients. The aforementioned methods for vitamin C testing and detection just serve as an example to illustrate how complicated and difficult for a general population to perform the testing by themselves. These testing needs non-house held chemicals, needs expensive instruments such as Infrared Spectrometers, Gas Chromatography and high performance liquid chromatography, needs well-equipped laboratories and requires well-trained professional skills. Therefore, a simple and easy-to-use testing for nutrients will greatly appreciated by those consumers who would like to validate the nutrients in their consumable products.

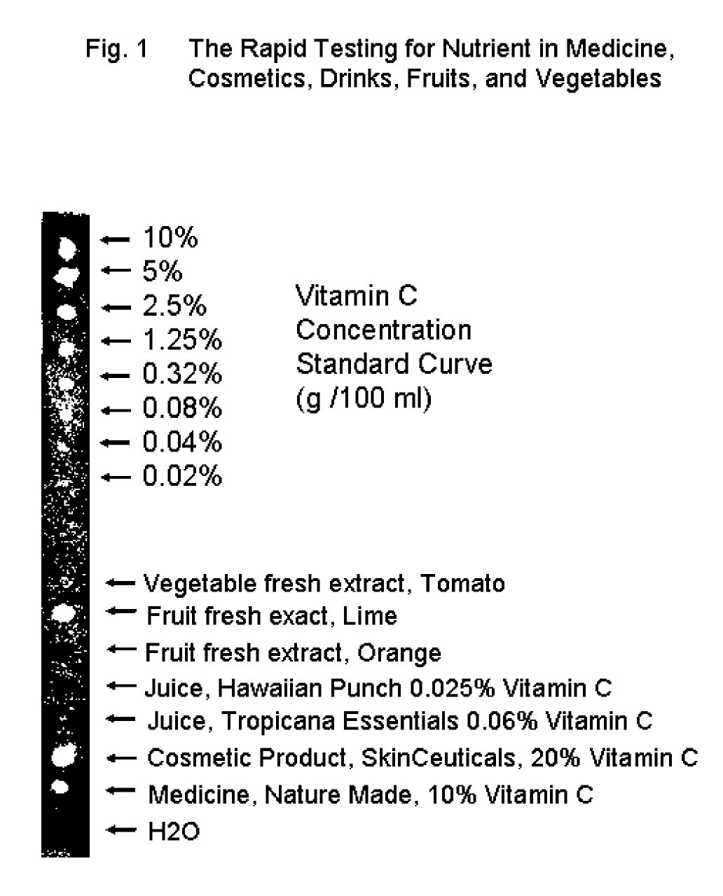

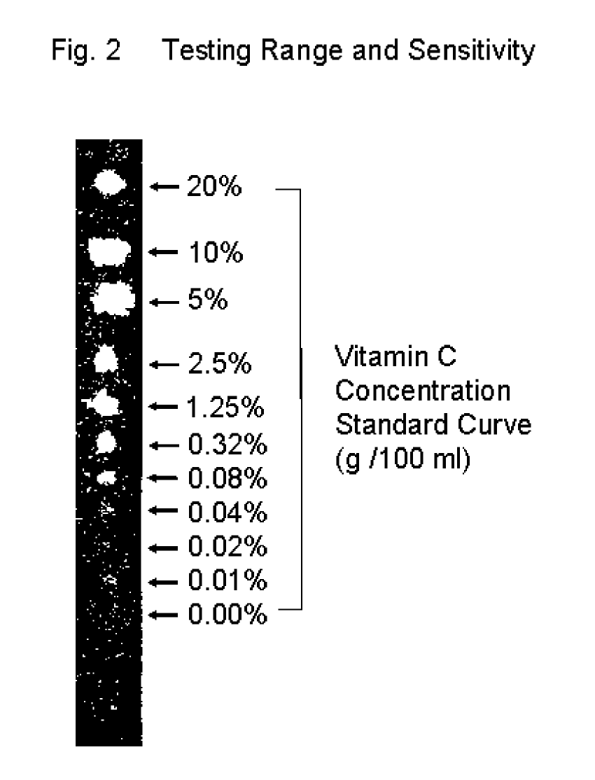

Present invention provides a rapid testing for nutrients by using strips that indicate the parameter of nutrients when contact with nutrients containing products. In one embodiment, the rapid testing strips can test nutrients in different products with broad testing range and high sensitivity. As shown in FIG. 1, vitamin C in medicine, cosmetic products, drinks, fruit and vegetable can be tested. It only needs 2 ul of liquid sample to perform the testing. The testing range of Vitamin C is from 20% to 0.02% as indicated in FIGS. 1 and 2. The sensitivity is about 0.4 ug. The testing range can go further lower and sensitivity can go even higher if use larger volume of liquid samples during the test.

In a preferred embodiment, using small volume of samples on strip testing will keep original color in the non-reacted area, which will increase the contrast between reacted area and non-reacted area. Therefore, the sensitivity of the testing is increased, and reading results by naked eye is more distinguishable and reliable. Using small volume of samples on strip testing also decreases the demanding for samples volume and avoids using sample holder. The large volume of samples can also be applied on the rapid testing strips. The testing strips can dip into samples containing nutrition, or samples can be poured onto the testing strips.

One additional embodiment of present invention is to apply electronic devices on the strip testing. The devices scan reacted area and non-reacted area to calculate accurately the amount of nutrients in the testing samples. The computer program in the devices performs background subtraction, relative signal differences between reacted area and non-reacted area, quantitation and analysis. The electronic devices are either on desk top or hand-held. The electronic devices can also be integrated into current existing testing instruments.

To develop simple and easy-to-use rapid testing for nutrients, strip as testing vehicle is invented as one of embodiment of this invention. Supporting materials of the strips for rapid testing can be plastic, paper, cotton, synthetic, natural or recycled fabric. Coating or pre-treatment of supporting materials may increase performance of the testing. Supporting materials can have color or colorless. The shape of rapid testing can be disc, ball, cube, strips, strap, stick, square, sheet, round, or patch. Indicators used on strips can have different color including, but not limited, red, blue, green, black, orange, yellow, purple. The indicators include, but not limited, synthetic dye, natural dye, food color, organic chemicals or non-organic chemicals. The rapid testing strips may contain single indicator or multiple indicators. The amount of indicators on the rapid testing strips is ranged broadly and is optimized according to testing performance.

### EXAMPLE 1

### Rapid Testing for Nutrients

Nutrients such as vitamin C in medicine, cosmetic products, juices, fruits and vegetable are tested with Rapid Testing invented herein. The medicine is vitamin C tablet containing 500 mg of vitamin C from Nature Made (Purchased from Costco); The cosmetic product containing 20% of vitamin C from SkinCeuticals Serum 20; The Juice 1 containing 0.06% of vitamin C from Tropicana Essentials (Purchased from Safeway); The Juice 2 containing 0.025% of vitamin C from Hawaiian Punch (Purchased from Safeway); The orange, lime and tomato were purchased from Safeway also. The vitamin C tablet was dissolved in 5 ml H20. The clear supernatant extract was used as testing samples; the original liquid from cosmetic product and juices was used as testing samples; squished juices from orange, lime and tomato were directly tested by rapid testing. The vitamin C in standard curve was diluted from pure vitamin C from Sigma. 2 micro liters of samples were applied on rapid testing strips. The result was read two minutes later.

As estimated data of rapid testing in FIG. 1, the vitamin C tablet and cosmetic product do contain correct amount of vitamin C; Tropicana Essentials contains more vitamin C than Hawaiian Punch; Lime contains large amount of vitamin C; to our surprise, even tomato contains more vitamin C than orange. We tasted the orange and it is a kind of sweetish, which may explain it because grade of maturity and storage condition of fruits will affect the amount of vitamin C contained in the fruits. These testing data were corresponding to the actual amount of vitamin C contained in the products respectively. In 100 g of products, orange contains 50 mg vitamin C, lime contains 40 mg vitamin C and tomato contains 10 mg vitamin C (http://en.wikipedia.org/wiki/Vitamin_C#Plant_sources Tomato contains 0.01% of vitamin C and Tropicana Essentials juice contains 0.06% of vitamin C. The testing data of tomato and Tropicana Essentials juice in rapid testing is very close to the vitamin C standard cure at 0.02%, 0.04% and 0.08% respectively as shown in FIG. 1. Therefore, rapid testing for nutrients in this invention is applicable for nutrient testing in most products.

### EXAMPLE 2

### Range and Sensitivity of Rapid Testing

The pure vitamin C from Sigma was serial diluted. Concentration from 20% to 0.01%. 2 micro liters of sample containing different concentration of vitamin C was applied on rapid testing. Result was read 2 minutes afterward as shown in FIG. 2. Sample containing 0.01% of vitamin C can be detected with rapid testing by naked eye. With electronic devices, the sensitivity can increased at least to 0.01% of vitamin C. The minimum detectable amounts of vitamin C in 2 micro liters are 0.2 micro grams by naked eye or less than 0.2 micro grams by electronic devices.

### EXAMPLE 3

### Validation of Nutrients in Commercial Products

Amount of vitamin C in five commercial available products for vitamin C supplement were validated by rapid testing. 1) The vitamin C tablet made by Nature Made contains 500 mg vitamin C (Nature Made); 2) Multi-vitamin tablet for Children made by Kirkland contains 250 mg vitamin C (Children); 3) Multi-vitamin tablet for adult made by Kirkland contains 120 mg vitamin C (Adult); 4) the One Day Women's multi-vitamin contains 60 mg vitamin C (Women); and 5) The Tropicana Essentials Juice contains 60 mg vitamin C per 100 ml juice (Juice).

The above four tablets containing vitamin C were dissolved in 5 ml water to make final vitamin C concentration at 10% (Nature Made); 5% (Children); 2.4% (Adult) and 1.2% (Women). The filtrates from each tablet were performed on rapid testing strip. The original Tropicana Essentials Juice containing 0.06% of vitamin C was directly applied on the same rapid testing strip. A serial diluted Standard Solution containing vitamin C at 5%, 2.5%, 1.25%, 0.63%, 0.32% and 0.16% were applied on the same testing strip to serve as standard curve for quantitation purpose.

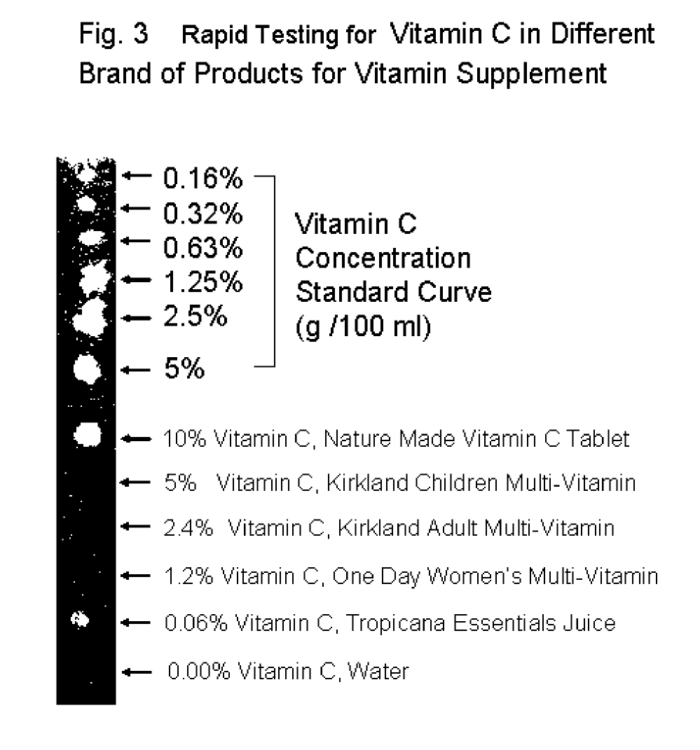

Validating results by rapid testing indicated that the vitamin C tablet made by Nature Made contains correct amount of vitamin C, whereas all three multi-vitamin tables did not contain correct amount of vitamin C as shown in FIG. 3. The Tropicana Essentials Juice contains correct amount of vitamin C.

### EXAMPLE 4

### Optimizing Rapid Testing for Nutrient

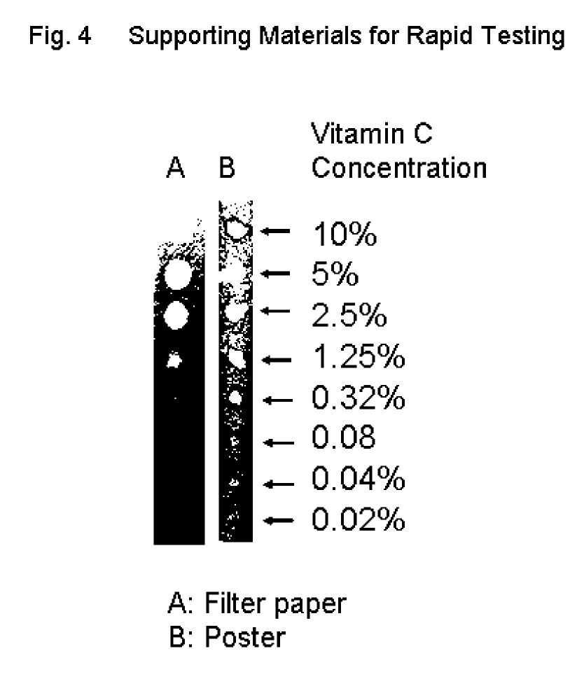

Supporting materials, indicator coated on supporting materials and volume of samples used on rapid strips can be optimized to achieve the best testing conditions. As show in FIG. 4, filter paper generates round and even spot than poster. The plastic sheet or synthetic fabrics can also be applied in this invention.

As an embodiment, the indicator coated on strips in this invention is methylthionine chloride, 3,7-bis(dimethylamino)phenothiazin-5-ium chloride or Methylene Blue. The molecular formula is C₁₆H₁₈ClN₃S. Its CAS number is 61-73-4: its EC number is 200-515-2. The methylthionine chloride was dissolved in ethanol at 0.001% to 5% (W/V) to make stock indicator solution. The stock indicator solution was diluted from 0 times to 100 times to make coating solution. The preferred concentration for stock indicator solution is depended on the supporting materials used. 0.05% of methylthionine chloride in stock solution was used in this invention when using filter paper as supporting material.

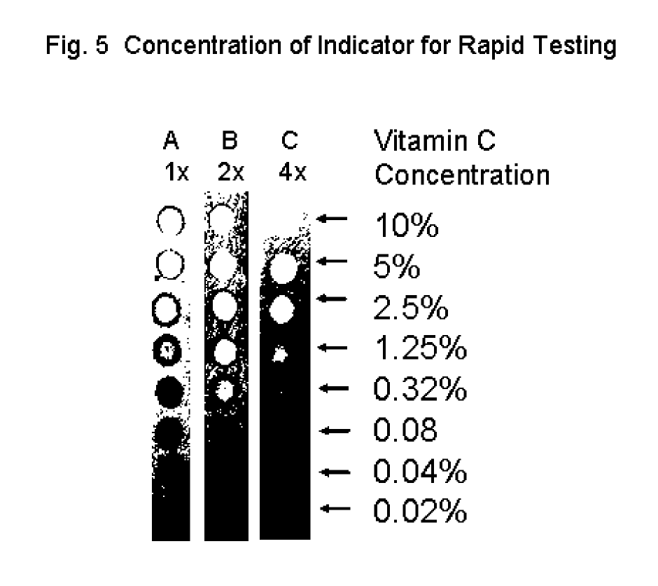

Dilution of stock indicator solution was also depended on the supporting marterials used and readout methods or devices. As indicated in FIG. 5, high concentration of indicator may not have the best readout at 1.25% of vitamin C, but may have highest sensitivity. As an embodiment in this invention, 4 times dilution from stock indicator solution was used to prepare the rapid testing for nutrient.

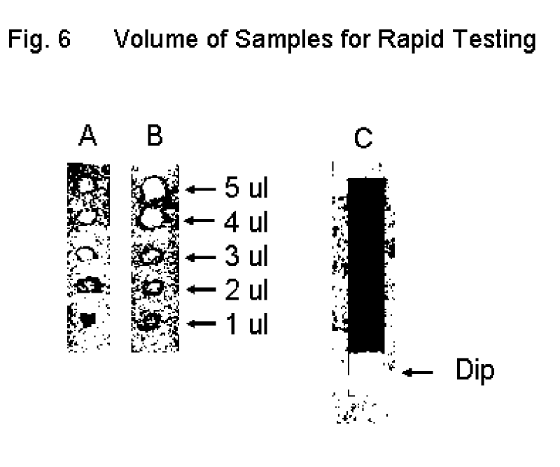

There are different ways to use the rapid testing for nutrient. The sample to be tested can be spotted on the testing strips, testing strips can be dipped in samples, or testing strips can be immersed in to samples. As an embodiment, spotting samples on testing strips or dipping testing strips into samples are preferred in this application because these two methods create a boundary between reacted area and unreacted area. As shown in FIG. 6, unreacted area will serve as background to increase the contrast of testing and therefore, increase the sensitivity and reliability of rapid testing. The boundary between reacted area and unreacted area can be recognized very well by electronic devices to make the rapid testing quantitative. Application of electronic devices makes the quantitation of the rapid testing more objective and accurate. These advantages are hardly be taken if the testing strips were immersed into samples.

### EXAMPLE 5

### Specificity of Rapid Testing for Nutrient

Common influencing substances such as acid, base and oxidizing substances were tested on rapid testing. Hydrochloride Acid (HCl) from Sigma is a strong acid and Citric Acid (CA) from Sigma is a weak acid existing in some fruits or vegetables. Sodium Hydroxide (NaOH) from Sigma is a strong base and Hydroperoxide (H2O2) from Fisher is most representative oxidizer. The acid and base were prepared at 100 mM to mimic the conditions naturally existed acidic or basic influencing substances. 30% Hydroperoxide was prepared to exaggerate conditions the potential oxidizing substances may create.

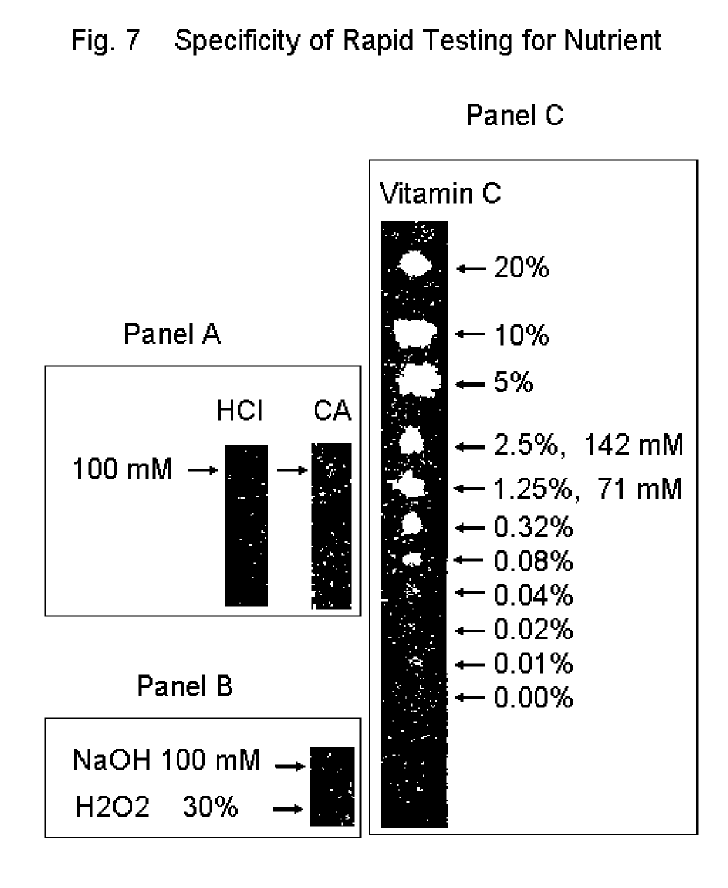

As shown in Panel A of FIG. 7, acidic substances such as HCl and Citri Acid only generate a very little signal in rapid testing comparing with the vitamin C at the similar concentration in Panel C. Basic substance such as NaOH and oxidizing substance such as and Hydroperoxide did not see any signal on rapid testing strips as shown in Panel B of FIG. 7. The above testing result indicated that the rapid testing for nutrient in this invention is predominantly specific to vitamin C. Acidic substance may cause a little noise and basic substances and oxidizing substances will not affect the specificity of the rapid testing for vitamin C in this invention.

The invention has been described using exemplary preferred embodiments. However, for those skilled in this field, the preferred embodiments can be easily adapted and modified to suit additional applications without departing from the spirit and scope of this invention. Thus, it is to be understood that the scope of the invention is not limited to the disclosed embodiments. On the contrary, it is intended to cover various modifications and similar arrangements based upon the same operating principle. The scope of the claims, therefore, should be accorded the broadest interpretations so as to encompass all such modifications and similar arrangements.

What is claimed is:

1. A method in the rapid testing for nutrient in products including, but not limited, medicine, cosmetics, drinks, fruits, and vegetables comprising the steps of
(1) Making the rapid testing by staining or coating supporting materials with indicators specifically reacting to nutrient;
(2) Applying small amount of samples from the products containing nutrient to a portion of indicator area on rapid testing made in step (1) or dipping a portion of indicator area on rapid testing made in step (1) into samples. Waiting 1 second to 1 hour for signal development from the portion of indicator area applied with the samples on testing strips made in step (1). A boundary between the portions of indicator area applied with the samples and without samples will form;
(3) Reading signal developed from step (2) by naked eyes or by electronic devices. Analyzing the difference in signal readout around boundary between the portions of indicator area applied with the samples on testing strips made in step (1) and the portion of indicator area without the samples on testing strips made in step (1). The amount of nutrient in the samples can be estimated by naked eyes or quantified by electronic devices.

2. The method in claim 1 the nutrient including, but not limited, vitamins, minerals, protein, carbohydrates, fat, nuclear acid, and any compounds that can apply to rapid testing.

3. The method in claim 1 the products are any products containing nutrient that can be made into appropriate format, such as liquid, for testing.

4. The method in step (1) of claim 1 the supporting materials include, but not limited, plastic, paper, cotton, synthetic, natural or recycled fabric. The supporting materials can be made into any shapes, such as disc, ball, cube, strips, strap, stick, square, sheet, round, or patch. Indicators used on strips can have different color including, but not limited, red, blue, green, black, orange, yellow, purple.

5. The method in step (1) of claim 1 the indicators include, but not limited, synthetic dye, natural dye, food color, organic chemicals or non-organic chemicals. The rapid testing may contain single indicator or multiple indicators. The amount of indicators for staining or coating the rapid testing is ranged broadly from 0.001% to 5% (W/V) and is optimized according to testing performance.

6. The method in step (2) of claim 1 the small amount of samples means small volume of samples. The volume of samples is relatively small enough to cover only a portion of indicator area on rapid testing made in step (1).

7. The method in step (2) of claim 1 a portion of indicator area on rapid testing means a part of whole area stained or coated with indicators, which continues evenly with rest part of whole area stained or coated with indicators without breaks or separation.

8. The method in step (2) of claim 1 a boundary was formed after signal development by testing samples. Different signal on the portions of indicator area applied with the samples and without the samples formed the contrasted boundary.

9. The method in step (3) of claim 1 the electronic devices can detect signal developed from testing samples electronically with repetitive and objective accuracy and precise. The electronic devices also can memorize and analyze quantitatively the signal from rapid testing for nutrient. The electronic devices are designed in many styles, such as hand-held, desk-top or integrated into existing instruments.

10. The method in step (3) of claim 1 the difference in signal readout is the signal difference between the samples reacting with indicator and indicators itself. The boundary region has high contrast for reliable and sensitive readout for signal difference.

11. A method the rapid testing for validating nutrients in commercial products includes, but not limited, nutrient supplements, multi-vitamin products, single vitamin products, vitamin-containing juice, and etc.

12. A method in claim 11 validating nutrient is to evaluate if the products contain the correct amount of nutrient claimed on the label of products.

13. A method in claim 1 and 11 the sensitivity of rapid testing is suitable to detect nutrient such as vitamin C in products including, but not limited, medicine, cosmetics, drinks, fruits, vegetables, nutrient supplements, multi-vitamin products, single vitamin products, and etc.

14. A method in claim 1 and 11 the specificity of rapid testing is suitable to detect nutrient such as vitamin C in products including, but not limited, medicine, cosmetics, drinks, fruits, vegetables, nutrient supplements, multi-vitamin products, single vitamin products, and etc.

15. A product in the rapid testing for nutrient in products including, but not limited, medicine, cosmetics, drinks, fruits, and vegetables comprising the steps of
(1) Making the rapid testing by staining or coating supporting materials with indicators specifically reacting to nutrient;
(2) Applying small amount of samples from the products containing nutrient to a portion of indicator area on rapid testing made in step (1) of claim 15 or dipping a portion of indicator area on rapid testing made in step (1) of claim 15 into samples. Waiting 1 second to 1 hour for signal development from the portion of indicator area applied with the samples on testing strips made in step (1) of claim 15. A boundary between the portions of indicator area applied with the samples and without samples will form;
(3) Reading signal developed from step (2) of claim 15 by naked eyes or by electronic devices. Analyzing the difference in signal readout around boundary between the portions of indicator area applied with the samples on testing strips made in step (1) of claim 14 and the portion of indicator area without the samples on testing strips made in step (1) claim 15. The amount of nutrient in the samples can be estimated by naked eyes or quantified by electronic devices.
